(19) United States
(12) Patent Application Publication
(10) Pub. No.: US 2008/0318841 A1
(43) Pub. Date: Dec. 25, 2008

(54) Method For Preparing a Factor H Concentrate and the Use Thereof in the Form of a Drug

(75) Inventors: Abdessadar Sami Chtourou, Elancourt (FR); Claudine Mazurier, Villeneuve D'Ascq (FR); Michel Poulle, Wavrin (FR); Bernadette Cauvin, Orchies (FR); Frederic Dhainault, Boissy Le Sec (FR)

Correspondence Address: MARSH, FISCHMANN & BREYFOGLE LLP, 8055 East Tufts Avenue, Suite 450, Denver, CO, 80237, US

(73) Assignee: Laboratoire Francais Du Fractionnement Et Des Biotechnologies S.A., Les Ulis (FR)

(21) Appl. No.: 12/095,949

(22) PCT Filed: Dec. 7, 2006
(86) PCT No.: PCT/FR06/02693

§ 371 Date: Aug. 26, 2008

(30) Foreign Application Priority Data
Dec. 7, 2005 (FR) 0512404

(51) Int. Cl. A61K 38/17; C07K 14/435; A61P 7/00; C07K 1/18

(52) U.S. Cl. 514/8; 530/380; 530/416

(57) ABSTRACT

The invention relates to the use of a factor H for producing a drug for treating Uremic Haemolytic Syndrome (UHS), to a method for purifying the factor H from a frozen fresh plasma and to a factor H concentrate obtainable by said method.

The invention relates to the use of a Factor H for making a drug for treatment of the Hemolytic Uremic Syndrome (HUS), to a method for purifying the Factor H from frozen fresh plasma and to the Factor H obtained by this method.

### FIELD OF THE INVENTION

The hemolytic uremic syndrome (HUS) is defined by the association of micro-angiopathic hemolytic anemia, thrombopenia and a renal affection. It is the main cause of acute renal failures in children of less than 3 years of age.

There exist two forms of HUS.

In its typical form, HUS occurs during the summer period after an episode of often blood-stained diarrhea. Typical HUS is secondary to an infection, in the majority of the cases, an infection by enteropathogenic Escherichia coli, in particular serotype 0157:H7, a producer of verotoxins.

Beside the typical form, certain patients have a different presentation. HUS atypical forms appear without prodromes and have a more chronic course frequently resulting in chronic renal failure. A typical HUS may occur at any age. It only amounts to 5% of the cases of HUS in children. The clinical signs of the syndrome are due to the development of platelet-rich microclots in small vessels. This particularly affects the glomerules of the kidney causing acute renal affection. A typical HUS may be sporadic but it is often familial. In both of these situations, the disease generally has a recurrent development by exacerbation. Its prognosis is low. Further, there exists a high risk of recurrence of the disease after renal transplantation, leading to rejection of the graft in most cases.

HUS may be associated with hypocomplementemia.

The complement plays an essential role in defending the organism against infectious agents and in the inflammatory process.

It comprises both plasma proteins, many different cell surface receptors, certain of them present on inflammatory cells and others on cells of the immune system, as well as membrane regulatory proteins which protect the host cells from self-attack.

The plasma proteins of the complement are about 20 in number and operate either as enzymes or as binding proteins or as regulators (inhibitors or activators).

The complement may be activated through two different routes: the conventional route and the alternative route.

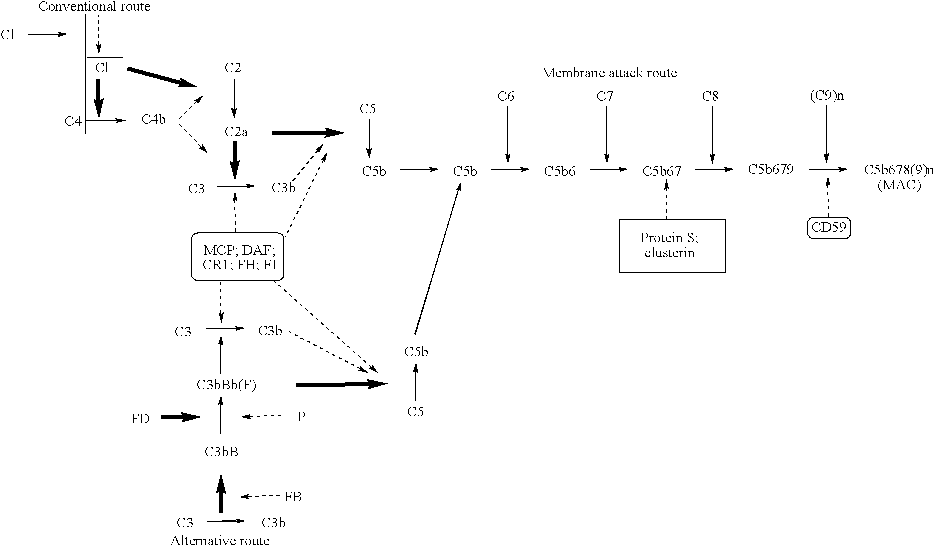

Enzymatic steps are illustrated by bold arrows. Regulatory proteins are framed: membrane proteins are in bold, circulating proteins in italics (to which belongs the Factor H noted as FH).

The conventional route is activated by antibodies binding to the foreign particle. It is therefore dependent on antibodies.

The alternative route is activated by the invasion of microorganisms; it is therefore independent of antibodies and extremely important in defending the host against bacterial infections.

The Factor H is a 155 kDa protein encountered in plasma at a concentration of 110-615 μg/mL. It is synthesized in the liver, the macrophages, the fibroblasts, the endothelial cells and platelets. The secreted form of the protein consists of 20 recurrent units of 60 amino acids. The Factor H is the central regulator of the alternative route of the complement. It is involved in the regulation of the rate of immune complexes in the blood and therefore in the equilibrium between the processes resulting in their generation or in their degradation.

With the Factor I, the Factor H inactivates the C3b molecules either free or bound to the surface of the cells. Thus, the immune complexes consisting of an antigen-antibody complex, complexed with the component of the C3b complement are no longer able to activate the subsequent cascade of the complement (components C5-C9).

The function of the Factor H may be broken down into three main activities:

1) The Factor H first of all behaves as a co-factor of the Factor I. Thus, the Factor H and the Factor I proceed with transforming the C3b protein of the complement into C3bi (inactive molecule) by cleaving the chain•of the protein C3b. The thereby inactivated protein C3b can no longer fulfill its role in the operation of the complement, and is no longer involved in forming the C3 convertase;

2) the Factor H is involved in the binding mechanisms to endothelial cells and to blood platelets;

3) finally the Factor H is involved in the dissociation of the preformed C3 convertase (C3bBb), in the alternative route of the activation of the complement. This latter activity directly depends on the molecular integrity of the Factor H, and proves to be more particularly dependent on the presence of an intact asn323-asn324 bond in the Factor H.

The deficiency or the absence of the Factor H, responsible for many cases of atypical HUS, therefore cause a hyperactivation of the complement, which is expressed in certain patients by the observation of deposits of C3 proteins during renal biopsies, and by a reduction of the C3 protein level present in the blood stream.

In certain patients affected by atypical HUS, the low C3 level is only observed during the acute phase of the disease. Strong arguments plead in favor of the role of a qualitative and quantitative Factor H deficiency often associated with a decrease in the level of C3, in the pathogeny of certain atypical HUS's (Rougier N. Kazatchkine M D, et al., Human complement factor H deficiency associated with hemolytic uremic syndrome, J. Am. Soc. Nephrol. 1998; 9:2318-2326).

Factor H deficiency is responsible for permanent activation of the alternative route of the complement responsible for a low level of C3.

A connection between the atypical HUS and a region coding for regulatory proteins of the complement, in particular the Factor H, located on the chromosome 1, has been demonstrated (Noris et al., Hypocomplementemia discloses genetic predisposition to hemolytic uremic syndrome and thrombotic thrombocytopenic purpura: role of Factor H abnormalities, J. Am. Soc. Nephrol. 1999, 10:281-293); (Warwicker et al., Genetic studies into hemolytic uremic syndrome, Kidney Int., 1998; 53:836-844); (Warwicker et al., Familial relapsing hemolytic uremic syndrome and complement Factor H deficiency, Nephrol. Dial. Transplant., 1999; 14:1229-1233).

The mutations of the gene of the Factor H were then identified in familial forms of HUS with recessive or dominant autosomal transmission (Buddles et al., Complement Factor H gene mutation associated with autosomal recessive atypical hemolytic uremic syndrome, Am. J. Hum. Genet., 2000; 66:1721-1722); (Caprioli et al, The molecular basis of familial hemolytic uremic syndrome: mutation analysis of Factor H gene reveals a hot spot in short consensus repeat 20, J. Am. Soc. Nephrol. 2001; 12:297-307); (Ohali et al., Hypocomplementemic autosomal recessive hemolytic uremic syndrome with decreased Factor H, Pediatr. Nephrol. 1998; 12:619-624); (Ying et al., Complement Factor H gene mutation associated with autosomal recessive atypical hemolytic uremic syndrome, Am. J. Hum. Genet. 1999; 65:1538-1546).

Recurrence after transplantation in patients having an atypical form of HUS is observed in about 25% of the cases. Prognosis in the case of recurrence is bad; loss of the graft related with recurrence is the rule.

### PRIOR ART

The first intention treatment consisting in perfusions of frozen fresh plasma with or without plasma exchanges was empirically undertaken in the 70's long before the role of the complement was known in HUS. Today, perfusions of frozen fresh plasma with or without plasma exchanges are basically used for HUS therapy. However, the amounts and the frequency of the perfusions of frozen fresh plasma are still determined empirically.

These perfusions should be repeated at regular intervals twice a week to twice a month, each perfusion lasting 2-3 hours.

This treatment is therefore long and recurrent for the patient.

The amounts of transfused frozen fresh plasma are significant, which increases the standard risks of frozen fresh plasma perfusion.

Firstly, frozen fresh plasma (FFP) contains anti-A or anti-B haemolysines and it should be reserved for patients with the same group ABO, or at the very least for patients lacking antigens A or B corresponding to haemolysines (a compatibility rule opposite to the one for red blood cells. Inobservation of these rules exposes the receiver to post-transfusional haemolysis of the red blood cells by ABO incompatibility.

Moreover, with the purpose of avoiding any risk of allo-immunization towards the antigen D of the Rhesus system, perfusions need to be carried out, above all in risk patients (girls, women of child bearing age, multi-transfused persons), where the patient and the donor have the same characteristics at the level of this antigen.

Secondly, FFP may cause hyperphosphatemia in HUS patients because the phosphate concentration in FFP, in particular in viro-attenuated plasma (VAP), is very high (9-12 mmol/L) and the HUS patient suffers from renal failure. The high phosphate concentration in VAP is likely to cause in patients transfused with VAP, hyperphosphatemia, all the more significant as:
the transfused VAP volumes are significant,
they are repeated daily,
renal failure pre-exists in the patient,
hyperphosphatemia pre-exists in the patient.

Next, the prefused amounts of FFP may cause a protein overload and/or a citrate overload which reduces the concentration of circulating calcium.

Finally, FFP causes a risk of allergies, as well as transmission of infectious agents. Indeed, present detection and inactivation methods do not always have sufficient sensitivity and inactivation capacity for allowing detection and removal of infectious agents potentially present in frozen fresh plasma.

The association of plasma exchanges with frozen fresh plasma perfusions is essential when the perfused volumes are too large for being removed by diuresis and for maintaining normal arterial pressure. This association has significant additional risks, mostly due to vascular access (requirement of a central route), to volume overload, to anaphylactic reaction, to problems of coagulation and to transmission of viral diseases.

Further, plasma exchanges are difficult to apply in young children.

Another treatment consists in kidney transplantation. However, the risk of recurrence after transplantation is very high.

Further, a diagnosis after renal transplantation in aHUS patients (atypical HUS) may be difficult. It may be difficult to distinguish between recurrence and an acute vascular rejection or a chronic rejection on a biopsy of the transplant.

Treatment of the recurrence consists in perfusions of frozen fresh plasma, plasma exchanges with or without plasma perfusions with very unpredictable results. These unpredictable results may be explained by the number and the volume of the FFP perfusions, each perfusion representing a pool of donations from several donors and not a homogenous batch.

As the Factor H is synthesized in the liver, it seems logical to propose a liver transplantation or even a combined liver-kidney transplantation.

This transplantation is always a difficult choice for physicians and parents and has operating risks and the risks of rejection of any liver transplantation.

### SUMMARY OF THE INVENTION

To find a remedy to these drawbacks of the prior art, the applicant surprisingly observed that it is possible to use the Factor H for making a drug intended for the treatment of HUS.

With the Factor H, for example as a Factor H concentrate derived from frozen fresh plasma, it is possible to restore deficiency of Factor H in patients affected by HUS while reducing the injected volumes and the injection times with a safe, stable and effective product.

In particular, by administering the Factor H in the period immediately after liver transplantation it is possible to compensate for the low Factor H production by the transplanted liver and thus for the immediate relapse and rejection of the graft.

The present invention also relates to a method for purifying the Factor H comprising the steps consisting in:

1) preparing the supernatant of a cryoprecipitate of plasma,

2) submitting this supernatant to chromatography on a gel/resin of the anion exchanger type,

3) submitting the non-retained fraction to chromatography on a gel/resin including a grafted ligand of the heparin type,

4) adjusting the pH of the non-retained fraction after chromatography of step 3 in order to allow binding of the Factor H to a chromatographic support gel/resin including a grafted ligand of the heparin type,

5) eluting the Factor H with a buffer of ionic force larger than that of the buffer for equilibrating the gel/resin,

6) diluting the eluted fraction, and then submitting it to chromatography on a gel/resin of the strong acid cation exchanger type,

7) eluting the Factor H with a buffer of ionic force larger than that of the buffer for equilibrating the gel/resin,

8) diluting the eluted fraction and then submitting it to chromatography on a gel/resin of the strong acid anion exchanger type,

9) washing the gel/resin and eluting the Factor H.

10) preparing a concentrate of Factor H.

### DETAILED DESCRIPTION OF THE INVENTION

Figures:

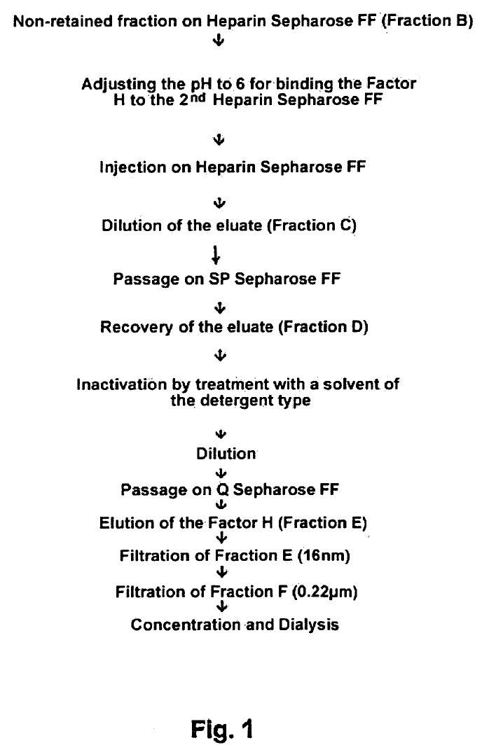

FIG. 1: Diagram of the method for purifying the Factor H

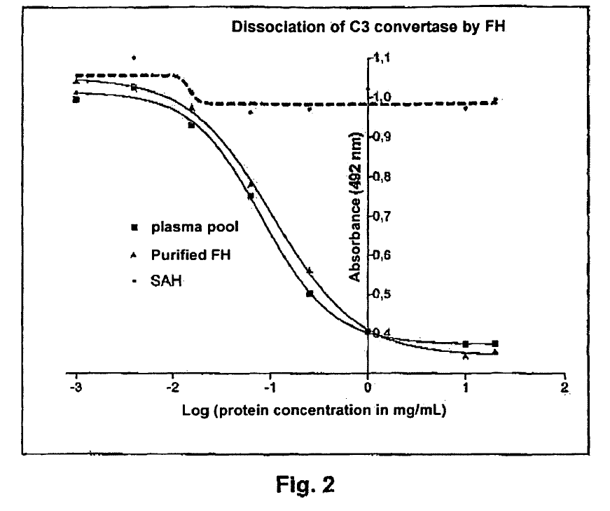

FIG. 2: Dissociation of C3 convertase by the Factor H.

The main object of the present invention is the use of the Factor H for making a drug intended for the treatment of Hemolytic Uremic Syndrome (HUS), in particular of the typical form of HUS or of the atypical form of HUS.

A preferred embodiment of the invention is the use of the Factor H for making a drug intended for the treatment of the hemolytic uremic syndrome, the Factor H being purified from fresh human plasma or plasma fractions stemming from purification by standard methods well known to one skilled in the art.

This purification is well known to one skilled in the art. It may occur by chromatography, using a column of lysine-sepharose, QAE-Sephadex, DEAE-Toyopearl, Sephacryl S-300 and hydroxyapatite.

It is detailed in the following documents: Fearon, J. Immunol. 119, 1248-1252 (1977); Crossley et al., Biochem. J., 191, 173-182, (1980); Nagasawa et al., J. Immunol., 125, 578-582, (1980); Weiler et al., P.N.A.S., 73, 3268-3272, (1976) and Whaley et al., J. Exp. Med., 144, 1147-1163 (1976).

The Factor H resulting from purification from frozen fresh plasma is for example found in the form of a Factor H concentrate.

Another embodiment of the invention is the use of the Factor H for making a drug intended for the treatment of hemolytic uremic syndrome, the Factor H being obtained by genetic engineering, by expressing its gene in a cell selected from the group consisting of bacteria, yeasts, fungi, or mammal cells.

A particular embodiment of the invention is the use of the Factor H for making a drug intended for the treatment of hemolytic uremic syndrome, the thereby obtained drug being in a freeze-dried form.

An additional embodiment of the invention consists in the use of the Factor H for making a drug intended for the treatment of hemolytic uremic syndrome, the thereby obtained drug having been subject to at least one method for removing or inactivating at least one infectious agent.

Among the infectious agents, mention may be made of viruses and non-conventional transmissible agents (NCTA) such as the prion protein.

In particular, the drug may be virally inactivated.

By <<virally inactivated >> is meant that the drug has been subject to at least one viral inactivation method known to one skilled in the art by treatment with chemicals, for example by solvent/detergent, and/or heat, for example by dry heating or pasteurization, and/or nanofiltration.

The viruses which may be inactivated by any of these methods comprise: the human immunodeficiency virus (HIV), the hepatitis A virus (HAV), the hepatitis B virus (HBV), the B19 parvovirus, the cytomegalovirus (CMV), the porcine parvovirus, the polio virus, the bovine viral diarrhea virus (BVDV), etc.

Another object of the invention is a freeze-dried and virally inactivated pharmaceutical composition for example as described above, and comprising Factor H and pharmaceutically acceptable excipients and/or carriers.

Another object of the present invention relates to a method for purifying the Factor H comprising the steps consisting in:

1) preparing the supernatant of a cryoprecipitate of plasma,

2) submitting this supernatant to chromatography on a gel/resin of the anion exchanger type,

3) submitting the non-retained fraction to chromatography on a gel/resin including a grafted ligand of the heparin type,

4) adjusting the pH of the non-retained fraction after chromatography of step 3 in order to allow binding of the Factor H to a chromatographic support gel/resin including a grafted ligand of the heparin type,

5) eluting the Factor H with a buffer of ionic force larger than that of the buffer for equilibrating the gel/resin,

6) diluting the eluted fraction, and then submitting it to chromatography on gel/resin of the strong acid cation exchanger type,

7) eluting the Factor H with a buffer of ionic force larger than that of the buffer for equilibrating the gel/resin,

8) diluting the eluted fraction, and then submitting it to chromatography on gel/resin of the strong acid anion exchanger type,

9) washing the gel/resin and eluting the Factor H,

10) preparing a concentrate of Factor H.

In a particular embodiment of the invention, the chromatographic support on which a heparin ligand for step 3) is grafted, is sepharose heparin gel/resin.

In a particular embodiment of the invention, the chromatographic support on which a heparin ligand for step 4) is grafted, is sepharose heparin gel/resin.

In a particular embodiment of the invention, the chromatography on gel/resin of the strong acid cation exchanger type of step 6) is a chromatography of SP sepharose type.

In a particular embodiment of the invention, the chromatography on a gel/resin of the strong acid anion exchanger type of step 8) is a chromatography of the Q sepharose FF type or equivalent.

Advantageously, the pH of the non-retained fraction of step 4) is adjusted so as to be comprised in the range from pH 5.5 to pH 6.5 and preferably so as to be equal to pH 6.0.

Advantageously, the pH of the diluted fraction in step 8) is adjusted so as to be comprised in the range from pH 6.5 to pH 7.5.

The purification method of the invention is the only known method for purifying a Factor H stemming from plasma which proves to be industrializable, and with which a purified Factor H concentrate may be obtained in the absence of inhibitors of chemical or synthetic proteases, therefore not leaving any trace of these inhibitors in the final product.

Indeed, the methods for purifying the Factor H from human plasma, known from the state of the art, are applied in a perspective of fundamental research, by sometimes using precipitation purification techniques (example PEG; ammonium sulfate) which are industrializable with difficulty, and protease inhibitors. These protease inhibitors inhibit the action of trypsin type proteins, present in serum and plasma, which are responsible for cleaving the protein bond joining the asn323 and asn324 amino acids of the Factor H molecule. Therefore, the addition of protease inhibitors contributes to reducing proteolysis of this factor and consequently improves its stability. However, the protease inhibitors are often highly toxic compounds, which make them unsuitable for an industrial method for producing a Factor H intended for therapeutic use.

Moreover, the method of the invention has a significant advantage in that a Factor H concentrate may be obtained, for which 3 types of main activities are retained, which none of the Factors H described in the state of the art has. The Factor H obtained by the method of the invention may therefore fulfill its activity of central regulator of the alternative route of the complement, an activity which proves to be deficient in patients affected by HUS, and notably by atypical HUS. In particular, the Factor H produced by the method of the invention retains its activity for dissociating the preformed C3 convertase in the alternative route of the complement and proves to be capable of being used in treating HUS by means of its full functional activity.

The Factor H concentrate obtained by the method of the invention further has a specific activity close to 1 (AS=0.8 to 0.9), which makes it more efficient than a solution of frozen fresh plasma (AS=0.008) which, although therapeutically effective, includes many disadvantages, as described in the introduction of the present application. Among these disadvantages, administration of plasma introduces into the organism unnecessary additional proteins for treating HUS (albumin, fibrinogen . . . ) which may on the other hand, trigger undesirable reactions related to protein overload or cause allergic reactions, known as <<serum disease >>.

Finally, inactivation of the transmissible viruses present in plasma proves to be generally more difficult and less performing than the one set up for inactivating the viruses present in blood derivatives. The Factor H concentrate obtained by the method of the invention may therefore benefit from recognized and tested treatments providing documented viral safety.

### EXAMPLES

### Example 1

### Method for Purifying the Factor H

The method applied for purifying the Factor H is illustrated schematically in FIG. 1.

Human frozen fresh plasma is unfrozen at a temperature between 1° C. and 6° C., and then the plasma supernatant of the cryoprecipitate is separated from the insoluble fraction of the cryoprecipitate by centrifugation.

The plasma supernatant of the obtained cryoprecipitate, the Factor H concentration of which is comprised in a range from about 400 to about 500 mg of Factor H/liter, is submitted to chromatography on a resin/gel of the anion exchanger type (for example, a gel/resin of the DEAE Sephadex type), in order to separate the Factors which depend on vitamin K, from the plasma supernatant by retaining these Factors on the resin/gel.

The non-retained plasma supernatant fraction (fraction A), the Factor H concentration of which is comprised in a range from about 400 to about 500 mg of Factor H/liter, is then subject to affinity chromatography on a gel/resin of the heparin sepharose FF type, in order to separate antithrombin III from this fraction A, by retaining antithrombin III on the resin/gel.

The pH of this non-retained fraction A (fraction B), the Factor H concentration of which is comprised in a range from about 300 to about 400 mg of Factor H/liter, is adjusted so as to be comprised in a range from pH 5.5 to pH 6.5, and preferably so as to be equal to pH 6.0.

The fraction B, for which the pH was adjusted, is subjected to chromatography on a second gel/resin of the heparin sepharose FF type or on any other chromatographic support including grafted ligands of the heparin type. Most proteins contained in the plasma fraction B are then eluted with the chromatography filtrate. The proteins weakly adsorbed on the gel/resin are removed by a series of washes and pre-elutions. The Factor H retained on the gel/resin is then eluted by using a buffer having an ionic force larger than that of the buffer used for equilibrating the gel/resin.

The eluted fraction containing the Factor H (fraction C) is diluted, and then submitted to chromatography on a gel/resin of the strong acid cation exchanger type, for example a gel/resin of the SP sepharose Ff type or equivalent. The proteins weakly adsorbed on the gel/resin are removed by a series of washes and pre-elutions. The Factor H retained on the gel/resin is then eluted by using a buffer having an ionic force larger than that of the buffer used of equilibrating the gel/resin.

The eluted fraction containing the Factor H (fraction D) is then submitted to a viral inactivation step by treatment with a solvent of the detergent type, for example Polysorbate 80 and TnBP. With such a treatment it is notably possible to efficiently inactivate the viruses, and in particular the viruses of the encapsulated type.

The fraction D is then diluted, and the pH of this fraction is adjusted so as to be comprised in a range from pH 6.5 to pH 7.5. The fraction D is then subject to chromatography on a gel/resin of the strong acid anion exchanger type, for example a gel/resin of the Q sepharose FF type or equivalent. After a series of washes, the Factor H retained on the gel/resin is eluted by using a buffer having an ionic force larger than that of the buffer used for equilibrating the gel/resin.

The agents introduced previously for achieving viral inactivation by treatment with a solvent of the detergent type are removed during this chromatographic step and the purity level of the Factor H is increased.

The eluted fraction containing the Factor H (fraction E) is then subject to a virus removal step by nanofiltration on a filter with a porosity of about 15 nm. This virus removal treatment provides efficient removal of the viruses, and in particular of non-encapsulated viruses of small size. The resulting solution (fraction F) is finally concentrated and adjusted by ultrafiltration and then filtered on a 0.22 μm filter.

The yield of the purification method described above and the specific activity of the Factor H purified by this method were measured on two distinct batches. The corresponding results are shown in Table 1. The specific activity (A.S.) is expressed in mg of antigen of Factor H type/mg of protein.

TABLE 1

| | Batch 1 | | Batch 2 | |
|---|---|---|---|---|
| Steps | Yield % | A.S. | Yield % | A.S. |
| Start | 100 | 0.008 | 100 | 0.005 |
| After heparin sepharose FF | 39.2 | 0.27 | 44 | 0.15 |
| After SP sepharose | 92.9 | 0.68 | 91 | 0.55 |
| After Q sepharose | 98.8 | 1.1 | 86.7 | 0.9 |
| After concentration | 88.6 | 0.98 | 90.7 | 0.87 |
| After filtration | 81.3 | 0.92 | 93.2 | 0.89 |

### Example 2

### Method for Dosing the Activity of the Factor H

The wells of an ELISA plate (of the 96-well type) are covered with a solution of purified C3b protein with a concentration of 2.5 ·g/mL (Calbiochem: ref. 341274) in a 0.2 M sodium carbonate buffer. To do this, 100 μL of solution are introduced into the wells and the plates are incubated for 1 hour at 37° C. and one night at 4° C.

Three washes of 300 μL/well are performed with a solution of 10 mM sodium phosphate buffer, 25 mM NaCl, 0.1% Tween 20 at pH 7.2.

The aspecific sites are then saturated by incubation for one hour at 37° C. with 300 μL/well of a solution of 10 mM sodium phosphate buffer, 25 mM NaCl, Tween0.05%, at pH 7.2, and containing 1% BSA. Next, a wash of the wells is performed with the washing solution described earlier.

100 μL of a solution containing:
75 μL of a 20 mM NiCl₂ mother solution (final concentration 1.5 mM);
4 μL of Factor B (Calbiochem ref. 341262) at a concentration of 1 mg/mL;
3 μL of Factor D (Calbiochem ref 341273) at a concentration of 1 mg/mL; and
918 μL of 10 mM sodium phosphate buffer, 25 mM NaCl, 4% BSA and at pH 7.2; are deposited in each well before proceeding with incubation for 2 hrs at 37° C.

Three successive washes of 300 μL/well are then performed with a solution of 10 mM sodium phosphate buffer, 25 mM NaCl, 0.1% Tween 20, at pH 7.2.

A range of Factor H solutions are prepared with respective Factor H concentrations of 20 μg/mL, 10 μg/mL, 1 μg/mL, 0.25 μg/mL, 0.0625 μg/mL, 0.015625 μg/mL, 0.00390625 μg/mL and 0.001 μg/mL. 100 μL of each solution are deposited in a different well and incubation for 30 min at 37° C. is carried out.

Three successive washes of 300 μL/well are then performed, with a solution of 10 mM sodium phosphate buffer, 25 mM NaCl, 0.1% Tween 20, at pH 7.2.

A goat anti-human factor B antibody solution (Calbiochem ref.: 341272) is diluted to 1/2,000 in a PBS buffer (Sigma P-3813), pH 7.4, containing 0.1% BSA, and then 100 μL of the diluted solution are deposited in the wells and incubation is performed for 1 hr at 37° C.

Three successive washes of 300 μL/well are performed with a solution of PBS, 0.1% Tween 20, at pH 7.2. Next 100 μL of a solution containing an anti-goat rabbit antibody labeled with peroxidase (Calbiochem ref. 401515, 1 mg/mL), and diluted to 1/10,000 in PBS containing 0.1% BSA, are then deposited in the wells in order to proceed with incubation for 20 to 25 minutes at room temperature.

Three successive washes of 300 μL/well are performed with a solution of PBS, 0.1% Tween 20.

The substrate of the OPD peroxidase (Sigma) at a concentration of 5 mg/10 mL in a sodium citrate solution, is added to the wells, as well as 10 μL of H₂O₂, finally in an amount of 100 μL/well. The reaction mixture is left in contact with the wells for about 10 minutes before proceeding with stopping the reaction by adding 50 μL of 4NH₂SO₄ per well.

The absorbance of the solution contained in the wells is then measured at a wavelength of 492 nm. The corresponding results are shown in FIG. 2. The graphic illustrations appearing in FIG. 2 give the value of the absorbance measured versus the Factor H concentration or versus the protein concentration (SAH).

A similar method for dosing the activity of the Factor H is described in the document, McRae et al., The Journal of Immunology, 2005, 174: 6250-6256.

1. The use of the Factor H for making a drug intended for the treatment of the Hemolytic Uremic Syndrome (HUS).

2. The use according to claim 1, characterized in that the drug is intended for the treatment of the typical form of HUS.

3. The use according to claim 1, characterized in that the drug is intended for the treatment of the atypical form of HUS.

4. The use according to claim 1, characterized in that said Factor H is purified from frozen fresh plasma or from a plasma fraction.

5. The use according to claim 1, characterized in that said Factor H is produced by genetic engineering by expressing the gene of the Factor H in a cell selected from the group consisting of bacteria, yeasts, fungi or mammal cells.

6. The use according to claim 1, characterized in that said drug is prepared in a freeze-dried form.

7. The use according to claim 1, characterized in that said drug has been subjected to at least one method for removing or inactivating at least one infectious agent.

8. The use according to claim 1, characterized in that said drug has been subjected to at least one method for viral inactivation.

9. A virally inactivated, freeze-dried pharmaceutical composition comprising Factor H and pharmaceutically acceptable excipients and/or carriers.

10. A method for purifying the Factor H comprising the steps:
1) preparing the supernatant of a cryoprecipitate of plasma,
2) submitting this supernatant to chromatography on a gel/resin of the anion exchanger type,
3) submitting the non-retained fraction to chromatography on a gel/resin including a grafted ligand of the heparin type,
4) adjusting the pH of the non-retained fraction after chromatography of step 3 in order to allow binding of the Factor H to a chromatographic support gel/resin including a grafted ligand of the heparin type,
5) eluting the Factor H with a buffer of an ionic force larger than that of the buffer for equilibrating the gel/resin,
6) diluting the eluted fraction, and then submitting it to chromatography on a gel/resin of the strong acid cation exchanger type,
7) eluting the Factor H with a buffer of an ionic force larger than that of the buffer for equilibrating the gel/resin,
8) diluting the eluted fraction, and then submitting it to chromatography on a gel/resin of the strong acid anion exchanger type,
9) washing the gel/resin and eluting the Factor H,
10) preparing a concentrate of Factor H.

11. The method according to claim 10, wherein the chromatographic support including a grafted ligand of the heparin type of step 3) is a heparin sepharose gel/resin.

12. The method according to claim 10, wherein the chromatographic support including a grafted ligand of the heparin type of step 4) is a heparine sepharose gel/resin.

13. The method according to claim 10, wherein the chromatography on a gel/resin of the strong acid cation exchanger type of step 6) is a chromatography of the SP sepharose type.

14. The method according to claim 10, wherein the chromatography on a gel/resin of the strong acid anion exchanger type of step 8) is a chromatography of the Q sepharose FF type or equivalent.

15. The method according to claim 10, wherein the pH of the non-retained fraction of step 4) is adjusted so as to be comprised in the range from pH 5.5 to pH 6.5 and preferably so as to be equal to pH 6.0.

16. The method according to claim 10, wherein the pH of the fraction diluted in step 8) is adjusted so as to be comprised in the range from pH6.5to pH 7.5.

17. A Factor H concentrate obtained by the method according to claim 10.

18. A Factor H concentrate obtained by the method according to claim 10, for use in the treatment of diseases resulting from deficient control of the activation of the complement.

19. A Factor H concentrate obtained by the method according to claim 10 for use in the treatment of the Hemolytic Uremic Syndrome (HUS).

20. A Factor H concentrate obtained by the method according to claim 10 for use in the treatment of the atypical form of the Hemolytic Uremic Syndrome (aHUS).

21. The use of a Factor H concentrate obtained by the method according to claim 10 for controlling activation of the complement in vitro or ex vivo.

22. The use of a Factor H concentrate obtained by the method according to claim 10 for obtaining a drug intended for the therapeutic or prophylactic treatment of diseases resulting from deficient control of the activation of the complement.

23. The use of a Factor H concentrate obtained by the method according to claim 10 for obtaining a drug intended for the therapeutic or prophylactic treatment of the Hemolytic Uremic Syndrome (HUS).

24. The use of a Factor H concentrate obtained by the method according to claim 10 for obtaining a drug intended for the therapeutic or prophylactic treatment of the atypical form of the Hemolytic Uremic Syndrome (aHUS).
